SEQUENCE LISTING (8 sequences)

SEQ ID NO: 1 - protein, 504 aa, Bifidobacterium adolescentis [sequence omitted]

SEQ ID NO: 2 - protein, 504 aa, artificial
  Modified Amino Acid (Modified version of SEQ. ID NO 1)
MKNKVQLITYADRLGDGTIKSMTDILRTRFDGVYDGVHILPFFTPFDGADAGFDPIDHTKVDERLGSWDDVAELSKTHNIMVDAIVNHMSWESKQFQDVLAKGEESEYYPMFLTMSSVFPNGATEEDLAGIYRPRPGLPFTAYKFAGKTRLVWVSFTPQQVDIDTDSDKGWEYLMSIFDQMAASHVSYIRLDAVGYGAKEAGTSCFMTPKTFKLISRLREEGVKRGLEILIEVHSYYKKQVEIASKVDRVYDFALPPLLLHALSTGHVEPVAHWTDIRPNNAVTVLDTHDGIGVIDIGSDQLDRSLKGLVPDEDVDNLVNTIHANTHGESQAATGAAASNLDLYQVNSTYYSALGCNDQHYIAARAVQFFLPGVPQVYYVGALAGKNDMELLRKTSNGRDINRHYYSTAEIDENLKRPVVKALNALAKFRNELDAFDGTFSYTTDDDTSISFTWRGETSQATLTFEPKRGLGVENTTPVAMLEWEDSAGDHRSDDLIANPPVVA

SEQ ID NO: 3 - protein, 504 aa, artificial
  Modified Amino Acid (Modified version of SEQ. ID NO 1)
MKNKVQLITYADRLGDGTIKSMTDILRTRFDGVYDGVHILPFFTPFDGADAGFDPIDHTKVDERLGSWDDVAELSKTHNIMVDAIVNHMSWESKQFQDVLAKGEESEYYPMFLTMSSVFPNGATEEDLAGIYRPRPGLPFTHYKFAGKTRYVWVSFTPQQVDIDTDSDKGWEYLMSIFDQMAASHVSYIRLDAVGYGAKEAGTSCFMTPKTFKLISRLREEGVKRGLEILIEVHSYYKKQVEIASKVDRVYDFALPPLLLHALSTGHVEPVAHWTDIRPNNAVTVLDTHDGIGVIDIGSDQLDRSLKGLVPDEDVDNLVNTIHANTHGESQAATGAAASNLDLYQVNSTYYSALGCNDQHYIAARAVQFFLPGVPQVYYVGALAGKNDMELLRKTSNGRDINRHYYSTAEIDENLKRPVVKALNALAKFRNELDAFDGTFSYTTDDDTSISFTWRGETSQATLTFEPKRGLGVENTTPVAMLEWEDSAGDHRSDDLIANPPVVA

SEQ ID NO: 4 - protein, 504 aa, artificial
  Modified Amino Acid (Modified version of SEQ. ID NO 1)
MKNKVQLITYADRLGDGTIKGMTDILRTRFDGVYDGVHILPFFTPFDGADAGFDPIDHTKVDERLGSWDDVAELSKTHNIMVDAIVNHMSWESKQFQDVLAKGEESEYYPMFLTMSSVFPNGATEEDLAGIYRPRPGLPFTHYKFAGKTRLVWVSFTPQQVDIDTDSDKGWEYLMSIFDQMAASGVSYIRLDAVGYGAKEAGTSCFMTPKTFKLISRLREEGVKRGLEILIEVHSYYKKQVEIASKVDRVYDFALPPLLLHALSTGHVEPVAHWTDIRPNNAVTVLDTHDGIGVVDIGSDQLDRSLKGLVPDEDVDNLVNTIHANTHGESQAATGAAASNLDLYQVNSTYYSALGCNDQHYIAARAVQFFLPGVPQVYYVGALAGKNDMELLRKTNNGRDINRHYYSTAEIDENLKRPVVKALNALAKFRNELDAFDGTFSYTTDDDTSISFTWRGETSQATLTFEPKRGLGVENTTPVAMLEWEDSAGDHRSDDLIANPPVVA

SEQ ID NO: 5 - protein, 504 aa, artificial
  Modified Amino Acid (Modified version of SEQ. ID NO 1)
MKNKVQLITYADRLGDGTIKSMTDILRTRFDGVYDGVHILPFFTPFDGADAGFDPIDHTKVDERLGSWDDVAELSKTHNIMVDAIVNHMSWESKQFQDVLAKGEESEYYPMFLTMSSVFPNGATEEDLAGIYRPRPGLPFTHYKFAGKTRLVWVSFTPQQVDIDTDSDKGWEYLMSIFDQMAASGVSYIRLDAVGYGAKEAGTSCFMTPKTFKLISRLREEGVKRGLEILIEVHSYYKKQVEIASKVDRVYDFALPPLLLHALSTGHVEPVAHWTDIRPNNAVTVLDTHDGIGVVDIGSDQLDRSLKGLVPDEDVDNLVNTIHANTHGESQAATGAAASNLDLYQVNSTYYSALGCNDQHYIAARAVQFFLPGVPQVYYVGALAGKNDMELLRKTNNGRDINRHYYSTAEIDENLKRPVVKALNALAKFRNELDAFDGTFSYTTDDDTSITFTWRGETSQATLTFEPKRGLGVENTTPVAMLEWEDSAGDHRSDDLIANPPVVA

SEQ ID NO: 6 - protein, 504 aa, artificial
  Modified Amino Acid (Modified version of SEQ. ID NO 1)
MKNKVQLITYADRLGDGTIKSMTDILRTRFDGVYDGVHILPFFTPFDGADAGFDPIDHTKVDERLGSWDDVAELSKTHNIMVDAIVNHMSWESKQFQDVLAKGEESEYYPMFLTMSSVFPNGATEEDLAGIYRPRPGLPFTHYKFAGKTRLVWVSFTPQQVDIDTDSDKGWEYLMSIFDQMAASGVSYIRLDAVGYGAKEAGTSCFMTPKTFKLISRLREEGVKRGLEILIEVHSYYKKQVEIASKVDRVYDFALPPLLLHALSTGHVEPVAHWTDIRPNNAVTVLDTHDGIGVVDIGSDQLDRSLKGLVPDEDVDNLVNTIHANTHGESQAATGAAASNLDLYQVNSTYYSALGCNDQHYIAARAVQFFLPGVPQVYYVGALAGKNDMELLRKTNNGRDINRHYYSTAEIDENLKRPVVKALNALAKFRNELDAFDGTFSYTTDDDTSISFTWRGETSQATLTFEPKRGLGVDNATPVAMLEWEDSAGDHRSDDLIANPPVVA

SEQ ID NO: 7 - protein, 504 aa, artificial
  Modified Amino Acid (Modified version of SEQ. ID NO 1)
MKNKVQLITYADRLGDGTIKSMTDILRTRFDGVYDGVHILPFFTPFDGADAGFDPIDHTKVDERLGSWDDVAELSKTHNIMVDAIVNHMSWESKQFQDVLAKGEESEYYPMFLTMSSVFPNGATEEDLAGIYRPRPGLPFTHYKFAGKTRLVWVSFTPQQVDIDTDSDKGWEYLMSIFDQMAASHVSYIRLDAVGYGAKEAGTSCFMTPKTFKLISRLREEGVKRGLEILIEVHSYYKKQVEIASKVDRVYDFALPPLLLHALSTGHVEPVAHWTDIRPNNAVTVLDTHDGIGVIDIGSDQLDRSLKGLVPDEDVDNLVNTIHANTHGESQAATGAAASNLDLYQVNSTYYSALGCNDQHYIAARAVQFFLPGVPQVYYVGALAGKNDMELLRKTSNGRDINRHYYSTAEIDENLKRPVVKALNALAKFRNELDAFDGTFSYTTDDDTSISFTWRGETSQATLTFEPKRGLGVENTTPVAMLEWEDSAGDHRSDDLIANPPVVA

SEQ ID NO: 8 - protein, 504 aa, artificial
  Modified Amino Acid (Modified version of SEQ. ID NO 1)
MKNKVQLITYADRLGDGTIKSMTDILRTRFDGVYDGVHILPFFTPFDGADAGFDPIDHTKVDERLGSWDDVAELSKTHNIMVDAIVNHMSWESKQFQDVLAKGEESEYYPMFLTMSSVFPNGATEEDLAGIYRPRPGLPFTHYKFAGKTRLVWVSFTPQQVDIDTDSDKGWEYLMSIFDQMAASGVSYIRLDAVGYGAKEAGTSCFMTPKTFKLISRLREEGVKRGLEILIEVHSYYKKQVEIASKVDRVYDFALPPLLLHALSTGHVEPVAHWTDIRPNNAVTVLDTHDGIGVVDIGSDQLDRSLKGLVPDEDVDNLVNTIHANTHGESQAATGAAASNLDLYQVNSTYYSALGCNDQHYIAARAVQFFLPGVPQVYYVGALAGKNDMELLRKTNNGRDINRHYYSTAEIDENLKRPVVKALNALAKFRNELDAFDGTFSYTTDDDTSISFTWRGETSQATLTFEPKRGLGVENTTPVAMLEWEDSAGDHRSDDLIANPPVVA

(19) United States
(12) Patent Application Publication
(10) Pub. No.: US 2020/0347424 A1
(43) Pub. Date: Nov. 5, 2020

(54) SACCHAROSE PHOSPHORYLASE

(71) Applicant: PFEIFER & LANGEN GMBH & Co. KG, Köln (DE)

(72) Inventors: Timo Johannes Koch, Elsdorf (DE); Thomas Hässler, Köln (DE); Birgit Brucher, Leipzig (DE); Andreas Vogel, Leipzig (DE)

(21) Appl. No.: 16/080,481

(22) PCT Filed: Mar. 7, 2017
(86) PCT No.: PCT/EP2017/055347

§ 371 Date: Aug. 28, 2018

(30) Foreign Application Priority Data
Mar. 8, 2016 (EP) 16159122.7

(51) Int. Cl. C12P 19/44; C12P 19/24

(52) U.S. Cl. CPC C12P 19/44; C12Y 204/0102; C12Y 204/01007; C12P 19/24

(57) ABSTRACT

The invention relates to a saccharose phosphorylase that catalyzes the synthesis of glucose-1-phosphate and fructose from saccharose and phosphate, among other things. The saccharose phosphorylase according to the invention can be considered to be a mutation of the saccharose phosphorylase from Bifidobacterium adolescentis. In comparison to wild-type saccharose phosphorylase, the saccharose phosphorylase according to the invention is distinguished by improved activity, process stability, temperature stability, and lower product inhibition and is therefore particularly suitable for use in industrial processes.

The invention relates to a sucrose phosphorylase which, inter alia, catalyzes the synthesis of glucose 1-phosphate and fructose from sucrose and phosphate. The sucrose phosphorylase according to the invention can be understood as mutants of the sucrose phosphorylase from Bifidobacterium adolescentis. Compared to the wild-type sucrose phosphorylase, the sucrose phosphorylase according to the invention is distinguished by an improved activity, process stability, temperature stability, and by a lower product inhibition, and is therefore especially suitable for use in industrial processes.

Sucrose phosphorylases catalyze the conversion of sucrose and phosphate to fructose and glucose 1-phosphate. Besides this phosphorolytic activity, the enzymes are also capable, under appropriate conditions, of catalyzing in reverse the synthesis of sucrose from glucose 1-phosphate and fructose with the release of phosphate.

Cellobiose is a natural disaccharide which forms the basic building block for cellulose. Cellobiose is becoming increasingly attractive for the food and feed sector. Cellobiose can be produced via chemical or enzymatic hydrolysis of cellulose. GB 2 438 573 describes a process for obtaining cellobiose, comprising the hydrolysis of cellulose, ultrafiltration to increase the cellobiose percentage in the sugar solution relative to other saccharides, and crystallization to subsequently purify the cellobiose.

An alternative to the production of cellobiose from cellulose is the synthesis of cellobiose using cellobiose phosphorylases. In this connection, glucose 1-phosphate, as an intermediate, can be obtained either by phosphorolysis of starch by means of an alpha-glucan phosphorylase or by phosphorolysis of sucrose by means of a sucrose phosphorylase.

EP 0 423 768 discloses a method for producing cellobiose starting from sucrose using a sucrose phosphorylase, a glucose isomerase and a cellobiose phosphorylase. The method comprises the following steps: (1) cleavage of the sucrose in the presence of orthophosphate under sucrose phosphorylase catalysis into glucose 1-phosphate and fructose; (2) isomerization of fructose into glucose under glucose isomerase catalysis; (3) synthesis of cellobiose from glucose and glucose 1-phosphate under cellobiose phosphorylase catalysis with elimination of orthophosphate; (4) partial work-up of the cellobiose from the reaction mixture and also recycling of a portion of the remaining orthophosphate-containing reaction mixture into step (1).

WO 2011/124538 A1 discloses a variant of the sucrose phosphorylase from Bifidobacterium adolescentis, which has increased stability and is enzymatically active at 60° C. for 16 hours. Furthermore, there is a description of methods which express the enzyme and use it for the synthesis of glucose 1-phosphate and fructose.

Verhaeghe et al., “Mapping the acceptor site of sucrose phosphorylase from Bifidobacterium adolescensis by alanine scanning”, Journal of Molecular Catalysis B: Enzymatic, Vol. 96, 12.01.2013, pages 81-88, describes the investigation of the wild type of the sucrose phosphorylase from Bifidobacterium adolescensis with respect to its affinity for various substrates. A disclosure is made of various points in the amino acid sequence of the sucrose phosphorylase that have an influence on substrate affinity.

Cerdobbel et al., “Increasing the thermostability of sucrose phosphorylase by a combination of sequence- and structure-based mutagenesis”, Protein Engineering Design and Selection, Vol. 24, No. 11, Nov. 11, 2011, pages 829-834, discloses a variant of the sucrose phosphorylase from Bifidobacterium adolescentis having six mutations in the amino acid sequence that has an improved temperature stability at 60° C.

The databases DATABASE UniProt, ID A0A087AXC6, DATABASE Geneseq, SEQ ID 323, DATABASE UniProt, ID A0A087CXQ8 and DATABASE UniProt, ID A0A087CMV6 disclose mutations in the amino acid sequences of the sucrose phosphorylase from Bifidobacterium adolescensis.

The key intermediate of these syntheses of cellobiose is glucose 1-phosphate. Glucose 1-phosphate is passed through in many biochemical processes as an intermediate and therefore plays a central role in metabolism.

One way of optimizing enzymes consists in the use of enzyme engineering, which is geared toward the development of variants of a starting enzyme having improved properties. Enzyme engineering has already been applied to a sucrose phosphorylase from Bifidobacterim adolescentis. WO 2011/124538 relates to a sucrose phosphorylase from Bifidobacterium adolescentis that is suitable as biocatalysat for the conversion of carbohydrates at elevated temperature.

There is a need for improved methods for producing glucose 1-phosphate on an industrial scale, preferably from sucrose and phosphate as starting material, so that said glucose 1-phosphate can then subsequently be used in further reactions, especially for the synthesis of cellobiose under catalysis by a cellobiose phosphorylase.

It is an object of the invention to provide a sucrose phosphorylase having improved properties. With respect to the synthesis of glucose 1-phosphate from sucrose and phosphate, the sucrose phosphorylase should, in comparison with known sucrose phosphorylases, ideally be distinguished by an improved activity and process stability, especially temperature stability, and by a lower reactant and product inhibition.

This object is achieved by the subject matter of the claims.

A first aspect of the invention concerns a sucrose phosphorylase comprising an amino acid sequence which has an identity in relation to the amino acid sequence according to SEQ ID NO:1 of at least 80%, at least 81%, at least 82%, at least 83% or at least 84%, more preferably at least 85%, at least 86%, at least 87%, at least 88% or at least 89%, yet more preferably at least 90%, at least 91%, at least 92%, at least 93% or at least 94%, most preferably at least 95%, at least 96%, at least 97% or at least 98%, and especially at least 98.5%, at least 99.0%, at least 99.4%, at least 99.6% or at least 99.8%, and which, in comparison with SEQ ID NO:1, comprises at least one amino acid mutation, preferably at least two, at least three or at least four amino acid mutations, independently of one another in each case,
in a sequence segment A) corresponding to positions 11 to 31 according to SEQ ID NO:1; and/or
in a sequence segment B) corresponding to positions 132 to 161 according to SEQ ID NO:1; and/or
in a sequence segment C) corresponding to positions 175 to 195 according to SEQ ID NO:1; and/or
in a sequence segment D) corresponding to positions 285 to 305 according to SEQ ID NO:1; and/or
in a sequence segment E) corresponding to positions 394 to 406 according to SEQ ID NO:1; and/or
in a sequence segment F) corresponding to positions 447 to 459 according to SEQ ID NO:1; and/or
in a sequence segment G) corresponding to positions 464 to 484 according to SEQ ID NO:1.

In a preferred embodiment, the sucrose phosphorylase according to the invention comprises an amino acid sequence which has an identity in relation to the amino acid sequence according to SEQ ID NO:1 of at least 80% and which, in comparison with SEQ ID NO:1,
comprises an amino acid mutation in a sequence segment C) corresponding to positions 175 to 195 according to SEQ ID NO:1; and
comprises an amino acid mutation in a sequence segment D) corresponding to positions 285 to 305 according to SEQ ID NO:1.

In a preferred embodiment, the sucrose phosphorylase according to the invention comprises an amino acid sequence which has an identity in relation to the amino acid sequence according to SEQ ID NO:1 of at least 80%, at least 81%, at least 82%, at least 83% or at least 84%, more preferably at least 85%, at least 86%, at least 87%, at least 88% or at least 89%, yet more preferably at least 90%, at least 91%, at least 92%, at least 93% or at least 94%, most preferably at least 95%, at least 96%, at least 97% or at least 98%, and especially at least 98.5%, at least 99.0%, at least 99.4%, at least 99.6% or at least 99.8%, and which, in comparison with SEQ ID NO:1, comprises at least one amino acid mutation, preferably at least two, at least three or at least four amino acid mutations, independently of one another in each case,
in a sequence segment A) corresponding to positions 11 to 31 according to SEQ ID NO:1; and/or
in a sequence segment C) corresponding to positions 175 to 195 according to SEQ ID NO:1; and/or
in a sequence segment D) corresponding to positions 285 to 305 according to SEQ ID NO:1; and/or
in a sequence segment G) corresponding to positions 464 to 484 according to SEQ ID NO:1.

It has been found that, surprisingly, improved sucrose phosphorylases are obtainable by amino acid mutations in certain sequence segments of the amino acid sequence of the sucrose phosphorylase from Bifidobacterium adolescentis (wild type, SEQ ID NO:1).

In comparison with the wild-type sucrose phosphorylase, the sucrose phosphorylase according to the invention is distinguished by an improved activity, process stability, temperature stability, and by a lower reactant and product inhibition, and is therefore especially suitable for use in industrial processes. The improved properties can lead to an improved space-time yield and an increased ratio of phosphorolysis to synthesis.

The sucrose phosphorylase according to the invention comprises an amino acid sequence which has an identity in relation to the amino acid sequence according to SEQ ID NO:1 of at least 80%, preferably at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99%.

According to the invention, “identity” is defined as the percentage of identical concordances between two comparative sequences with optimal alignment. For optimal alignment, gaps can be inserted in either of the two sequences. Preference is given to determining the identity between two comparative sequences using the Smith and Waterman algorithm (Smith T F, Waterman M S (1981) J Mol. Biol. 147, 195-197), preferably using the computer program WATER from the EMBOSS package, which is freely available and has implemented the Smith and Waterman algorithm (Reis P, Longden I, Bleasby A (2000) Trends in Genetics 16, 276-277). Preference is given here to using BLOSUM62 for the substitution matrix with a GOP (gap opening penalty) of 10 and a GEP (gap extension penalty) of 0.5.

An amino acid mutation in the context of this invention is defined as exchanging the amino acid of a sucrose phosphorylase wild-type sequence, preferably of the sucrose phosphorylase wild-type sequence according to SEQ ID NO:1, into a different proteinogenic amino acid.

In preferred embodiments of the sucrose phosphorylase according to the invention,
sequence segment A) corresponds to positions 16 to 26 according to SEQ ID NO:1; and/or
sequence segment B) corresponds to positions 137 to 156 according to SEQ ID NO:1; and/or
sequence segment C) corresponds to positions 180 to 190 according to SEQ ID NO:1; and/or
sequence segment D) corresponds to positions 290 to 300 according to SEQ ID NO:1; and/or
sequence segment E) corresponds to positions 394 to 401 according to SEQ ID NO:1; and/or
sequence segment F) corresponds to positions 447 to 456 according to SEQ ID NO:1; and/or
sequence segment G) corresponds to positions 469 to 481 according to SEQ ID NO:1.

In particularly preferred embodiments of the sucrose phosphorylase according to the invention,
sequence segment A) corresponds to positions 20 to 22 according to SEQ ID NO:1; and/or
sequence segment B) corresponds to positions 141 to 152 according to SEQ ID NO:1; and/or
sequence segment C) corresponds to positions 184 to 186 according to SEQ ID NO:1; and/or
sequence segment D) corresponds to positions 294 to 296 according to SEQ ID NO:1; and/or
sequence segment E) corresponds to positions 395 to 397 according to SEQ ID NO:1; and/or
sequence segment F) corresponds to positions 450 to 452 according to SEQ ID NO:1; and/or
sequence segment G) corresponds to positions 473 to 477 according to SEQ ID NO:1.

In a preferred embodiment, the sucrose phosphorylase according to the invention, in comparison with SEQ ID NO:1, comprises at least one, but also more preferably not more than one, amino acid mutation in the sequence segment A) as defined above.

In a preferred embodiment, the sucrose phosphorylase according to the invention, in comparison with SEQ ID NO:1, comprises at least one, more preferably at least 2, but also yet more preferably not more than two, amino acid mutations in the sequence segment B) as defined above.

In a preferred embodiment, the sucrose phosphorylase according to the invention, in comparison with SEQ ID NO:1, comprises at least one, but also more preferably not more than one, amino acid mutation in the sequence segment C) as defined above.

In a preferred embodiment, the sucrose phosphorylase according to the invention, in comparison with SEQ ID NO:1, comprises at least one, but also more preferably not more than one, amino acid mutation in the sequence segment D) as defined above.

In a preferred embodiment, the sucrose phosphorylase according to the invention, in comparison with SEQ ID NO:1, comprises at least one, but also more preferably not more than one, amino acid mutation in the sequence segment E) as defined above.

In a preferred embodiment, the sucrose phosphorylase according to the invention, in comparison with SEQ ID NO:1, comprises at least one, but also more preferably not more than one, amino acid mutation in the sequence segment F) as defined above.

In a preferred embodiment, the sucrose phosphorylase according to the invention, in comparison with SEQ ID NO:1, comprises at least one, more preferably at least 2, but also yet more preferably not more than two, amino acid mutations in the sequence segment G) as defined above.

In preferred embodiments, the sucrose phosphorylase according to the invention, in comparison with SEQ ID NO:1, comprises more than one amino acid mutation, preferably at least two, three or four amino acid mutations.

In a preferred embodiment, the at least two, three or four amino acid mutations are, independently of one another, in sequence segment A), B), C), D), E), F) and/or G).

In a preferred embodiment, the at least two, three or four amino acid mutations are, independently of one another, in sequence segments A), C), D) and/or G), preference being given to one amino acid mutation in sequence segment A), one amino acid mutation in sequence segment C), one amino acid mutation in sequence segment D) and/or one amino acid mutation in sequence segment G).

In another preferred embodiment, the at least two, three or four amino acid mutations are, independently of one another, in sequence segments C), D), F) and/or G), preference being given to one amino acid mutation in sequence segment C), one amino acid mutation in sequence segment D), one amino acid mutation in sequence segment F) and/or one amino acid mutation in sequence segment G).

In preferred embodiments, the sucrose phosphorylase according to the invention, in comparison with SEQ ID NO:1, comprises at least two amino acid mutations, namely “first amino acid mutation in sequence segment”/ /“second amino acid mutation in sequence segment”, selected from the group consisting of: A/ /A, A/ /B, A/ /C, A/ /D, A/ /E, A/ /F, A/ /G; B/ /B, B/ /C, B/ /D, B/ /E, B/ /F, B/ /G; C/ /C, C/ /D, C/ /E, C/ /F, C/ /G; D/ /D, D/ /E, D/ /F, D/ /G; E/ /E, E/ /F, E/ /G; F/ /F, F/ /G; and G/ /G. In this connection, “E/ /F”, for example, means that the first of the at least two amino acid mutations is in the sequence segment E) and the second of the at least two amino acid mutations is in the sequence segment F).

In particularly preferred embodiments, the sucrose phosphorylase according to the invention, in comparison with SEQ ID NO:1, comprises at least two amino acid mutations, namely “first amino acid mutation in sequence segment”/ /“second amino acid mutation in sequence segment”, selected from the group consisting of: A/ /C, A/ /D, A/ /G; B/ /E, B/ /G; C/ /D, C/ /F, C/ /G; D/ /F, D/ /G; E/ /G; and F/ /G.

In particularly preferred embodiments of the sucrose phosphorylase according to the invention, the at least one, at least two, at least three or at least four amino acid mutation(s) is/are selected from the group consisting of
S21;
H142, L151;
H185;
I295;
N396;
S451;
D474, and T476.

Particularly preferred sucrose phosphorylases according to the invention comprise at least one, at least two or at least three amino acid mutations selected from the group consisting of H185, I295 and D474.

A particularly preferred sucrose phosphorylase according to the invention comprises one or two amino acid mutations selected from the group consisting of N396 and D474.

A particularly preferred sucrose phosphorylase according to the invention comprises one, two or three amino acid mutations selected from the group consisting of H142, N396 and D474.

A particularly preferred sucrose phosphorylase according to the invention comprises one, two or three amino acid mutations selected from the group consisting of L151, N396 and D474.

A particularly preferred sucrose phosphorylase according to the invention comprises one, two or three amino acid mutations selected from the group consisting of H185, I295 and T476.

A particularly preferred sucrose phosphorylase according to the invention comprises one, two or three amino acid mutations selected from the group consisting of H185, I295 and D474.

A particularly preferred sucrose phosphorylase according to the invention comprises one, two, three or four amino acid mutations selected from the group consisting of S21, H185, I295 and D474.

A particularly preferred sucrose phosphorylase according to the invention comprises one, two, three or four amino acid mutations selected from the group consisting of H185, I295, S451 and D474.

Preferably, the sucrose phosphorylase according to the invention, in comparison with SEQ ID NO:1, does not have certain amino acid mutations. Thus, it is preferred according to the invention that the sucrose phosphorylase according to the invention does not comprise at least one amino acid mutation selected from the group consisting of Q331E, R393N, D445P, D446G, D446T, Q460E and E485H, preferably does not comprise all these amino acid mutations.

In particularly preferred embodiments of the sucrose phosphorylase according to the invention, the at least one, at least two, at least three or at least four amino acid mutation(s) is/are selected from the group consisting of
S21G;
H142A, L151Y;
H185G;
I295V;
N396S;
S451T;
D474E, and T476A.

Particularly preferred sucrose phosphorylases according to the invention comprise at least one, at least two or at least three amino acid mutations selected from the group consisting of H185G, I295V and D474E.

A particularly preferred sucrose phosphorylase according to the invention comprises one or two amino acid mutations selected from the group consisting of N396S and D474E.

A particularly preferred sucrose phosphorylase according to the invention comprises one, two or three amino acid mutations selected from the group consisting of H142A, N396S and D474E.

A particularly preferred sucrose phosphorylase according to the invention comprises one, two or three amino acid mutations selected from the group consisting of L151Y, N396S and D474E.

A particularly preferred sucrose phosphorylase according to the invention comprises one, two or three amino acid mutations selected from the group consisting of H185G, I295V and T476A.

A particularly preferred sucrose phosphorylase according to the invention comprises one, two or three amino acid mutations selected from the group consisting of H185G, I295V and D474E.

A particularly preferred sucrose phosphorylase according to the invention comprises one, two, three or four amino acid mutations selected from the group consisting of S21G, H185G, I295V and D474E.

A particularly preferred sucrose phosphorylase according to the invention comprises one, two, three or four amino acid mutations selected from the group consisting of H185G, I295V, S451T and D474E.

Particularly preferably, the sucrose phosphorylase according to the invention comprises an amino acid sequence selected from the group consisting of SEQ ID NO: 2, 3, 4, 5, 6, 7 and 8.

In a preferred embodiment, the sucrose phosphorylase according to the invention comprises an amino acid sequence which differs from SEQ ID NO:1 and which has an identity in relation to the amino acid sequence according to SEQ ID NO:2 of at least 90%, at least 91%, at least 92%, at least 93%, at least 94%, at least 95%, at least 96%, at least 97%, at least 98% or at least 99%. Preferably, said amino acid sequence, in comparison with SEQ ID NO:1, has one or more amino acid mutations in the following positions, preferably all thereof: H142, N396 and D474. Preferably, said amino acid sequence, in comparison with SEQ ID NO:1, has one or more of the following amino acid mutations, preferably all thereof: H142A, N396S and D474E.

In a preferred embodiment, the sucrose phosphorylase according to the invention comprises an amino acid sequence which differs from SEQ ID NO:1 and which has an identity in relation to the amino acid sequence according to SEQ ID NO:3 of at least 90%, at least 91%, at least 92%, at least 93%, at least 94%, at least 95%, at least 96%, at least 97%, at least 98% or at least 99%. Preferably, said amino acid sequence, in comparison with SEQ ID NO:1, has one or more amino acid mutations in the following positions, preferably all thereof: L151, N396 and D474. Preferably, said amino acid sequence, in comparison with SEQ ID NO:1, has one or more of the following amino acid mutations, preferably all thereof: L151Y, N396S and D474E.

In a preferred embodiment, the sucrose phosphorylase according to the invention comprises an amino acid sequence which differs from SEQ ID NO:1 and which has an identity in relation to the amino acid sequence according to SEQ ID NO:4 of at least 90%, at least 91%, at least 92%, at least 93%, at least 94%, at least 95%, at least 96%, at least 97%, at least 98% or at least 99%. Preferably, said amino acid sequence, in comparison with SEQ ID NO:1, has one or more amino acid mutations in the following positions, preferably all thereof: S21, H185, I295 and D474. Preferably, said amino acid sequence, in comparison with SEQ ID NO:1, has one or more of the following amino acid mutations, preferably all thereof: S21G, H185G, I295V and D474E.

In a preferred embodiment, the sucrose phosphorylase according to the invention comprises an amino acid sequence which differs from SEQ ID NO:1 and which has an identity in relation to the amino acid sequence according to SEQ ID NO:5 of at least 90%, at least 91%, at least 92%, at least 93%, at least 94%, at least 95%, at least 96%, at least 97%, at least 98% or at least 99%. Preferably, said amino acid sequence, in comparison with SEQ ID NO:1, has one or more amino acid mutations in the following positions, preferably all thereof: H185, I295, S451 and D474. Preferably, said amino acid sequence, in comparison with SEQ ID NO:1, has one or more of the following amino acid mutations, preferably all thereof: H185G, I295V, S451T and D474E.

In a preferred embodiment, the sucrose phosphorylase according to the invention comprises an amino acid sequence which differs from SEQ ID NO:1 and which has an identity in relation to the amino acid sequence according to SEQ ID NO:6 of at least 90%, at least 91%, at least 92%, at least 93%, at least 94%, at least 95%, at least 96%, at least 97%, at least 98% or at least 99%. Preferably, said amino acid sequence, in comparison with SEQ ID NO:1, has one or more amino acid mutations in the following positions, preferably all thereof: H185, I295 and T476. Preferably, said amino acid sequence, in comparison with SEQ ID NO:1, has one or more of the following amino acid mutations, preferably all thereof: H185G, I295V and T476A.

In a preferred embodiment, the sucrose phosphorylase according to the invention comprises an amino acid sequence which differs from SEQ ID NO:1 and which has an identity in relation to the amino acid sequence according to SEQ ID NO:7 of at least 90%, at least 91%, at least 92%, at least 93%, at least 94%, at least 95%, at least 96%, at least 97%, at least 98% or at least 99%. Preferably, said amino acid sequence, in comparison with SEQ ID NO:1, has one or more amino acid mutations in the following positions, preferably all thereof: N396 and D474. Preferably, said amino acid sequence, in comparison with SEQ ID NO:1, has one or more of the following amino acid mutations, preferably all thereof: N396S and D474E.

In a preferred embodiment, the sucrose phosphorylase according to the invention comprises an amino acid sequence which differs from SEQ ID NO:1 and which has an identity in relation to the amino acid sequence according to SEQ ID NO: 8 of at least 90%, at least 91%, at least 92%, at least 93%, at least 94%, at least 95%, at least 96%, at least 97%, at least 98% or at least 99%. Preferably, said amino acid sequence, in comparison with SEQ ID NO:1, has one or more amino acid mutations in the following positions, preferably all thereof: H185, I295 and D474. Preferably, said amino acid sequence, in comparison with SEQ ID NO:1, has one or more of the following amino acid mutations, preferably all thereof: H185G, I295V and D474E.

Typically, the sucrose phosphorylase according to the invention catalyzes the conversion of sucrose and phosphate to glucose 1-phosphate and fructose. In this connection, the sucrose phosphorylase according to the invention, in comparison with the sucrose phosphorylase according to SEQ ID NO:1, preferably has

(i) an increased activity with respect to the conversion of sucrose and phosphate to glucose 1-phosphate and fructose in the presence of equimolar amounts of glucose 1-phosphate and fructose; and/or
(ii) a lower inhibition by fructose with respect to the conversion of sucrose and phosphate to glucose 1-phosphate and fructose in the presence of equimolar amounts of glucose 1-phosphate and fructose; and/or
(iii) a higher temperature stability after incubation at 58° C. for 15 min.

The following table juxtaposes the differences of these amino acid sequences according to the invention in comparison with the sucrose phosphorylase from Bifidobacterium adolescentis (wild type (WT), SEQ ID NO:1):

| SEQ ID | No. | A | B | | C | D | E | F | G | |
|---|---|---|---|---|---|---|---|---|---|---|
| NO: | mutations | S21 | H142 | L151 | H185 | 1295 | N396 | S451 | D474 | T476 |
| 1 | 0 | | | | | | | | | |
| 2 | 3 | | A | | | | S | | E | |
| 3 | 3 | | | Y | | | S | | E | |
| 4 | 4 | G | | | G | V | | | E | |
| 5 | 4 | | | | G | V | | T | E | |
| 6 | 3 | | | | G | V | | | | A |
| 7 | 2 | | | | | | S | | E | |
| 8 | 3 | | | | G | V | | | E | |

A further aspect of the invention concerns the use of the above-described sucrose phosphorylase according to the invention for the enzymatically catalyzed conversion of sucrose and phosphate to glucose 1-phosphate and fructose. Preferably, said use according to the invention subsequently comprises the reaction of the glucose 1-phosphate thus obtained with glucose to form cellobiose under enzymatic catalysis by a cellobiose phosphorylase.

All the preferred embodiments which were described above in connection with the sucrose phosphorylase according to the invention also apply correspondingly to its use according to the invention and are therefore not repeated at this point.

A further aspect of the invention concerns a method for producing glucose 1-phosphate and fructose, comprising the conversion of sucrose and phosphate under enzymatic catalysis by the above-described sucrose phosphorylase according to the invention.

All the preferred embodiments which were described above in connection with the sucrose phosphorylase according to the invention or with its use according to the invention also apply correspondingly to the method according to the invention and are therefore not repeated at this point.

A further aspect of the invention concerns a method for producing cellobiose and fructose from sucrose and glucose, comprising the steps of

(a) synthesis of glucose 1-phosphate and fructose by conversion of sucrose and phosphate under enzymatic catalysis by the above-described sucrose phosphorylase according to the invention;
(b) synthesis of cellobiose and phosphate by reaction of the glucose 1-phosphate with glucose under enzymatic catalysis by a cellobiose phosphorylase.

All the preferred embodiments which were described above in connection with the sucrose phosphorylase according to the invention or with its use according to the invention also apply correspondingly to the method according to the invention and are therefore not repeated at this point.

Further preferred embodiments are:

### Embodiment 1

Sucrose phosphorylase comprising an amino acid sequence which has an identity in relation to the amino acid sequence according to SEQ ID NO:1 of at least 80% and which, in comparison with SEQ ID NO:1, comprises at least one amino acid mutation
in a sequence segment A) corresponding to positions 11 to 31 according to SEQ ID NO:1; and/or
in a sequence segment B) corresponding to positions 132 to 161 according to SEQ ID NO:1; and/or
in a sequence segment C) corresponding to positions 175 to 195 according to SEQ ID NO:1; and/or
in a sequence segment D) corresponding to positions 285 to 305 according to SEQ ID NO:1; and/or
in a sequence segment E) corresponding to positions 394 to 406 according to SEQ ID NO:1; and/or
in a sequence segment F) corresponding to positions 447 to 459 according to SEQ ID NO:1; and/or
in a sequence segment G) corresponding to positions 464 to 484 according to SEQ ID NO:1.

### Embodiment 2

The sucrose phosphorylase according to embodiment 1, which has an identity in relation to the amino acid sequence according to SEQ ID NO:1 of at least 80% and which, in comparison with SEQ ID NO:1, comprises at least one amino acid mutation
in a sequence segment A) corresponding to positions 11 to 31 according to SEQ ID NO:1; and/or
in a sequence segment C) corresponding to positions 175 to 195 according to SEQ ID NO:1; and/or
in a sequence segment D) corresponding to positions 285 to 305 according to SEQ ID NO:1; and/or
in a sequence segment G) corresponding to positions 464 to 484 according to SEQ ID NO:1.

### Embodiment 3

The sucrose phosphorylase according to embodiment 1 or 2, wherein
sequence segment A) corresponds to positions 16 to 26 according to SEQ ID NO:1; and/or
sequence segment B) corresponds to positions 137 to 156 according to SEQ ID NO:1; and/or
sequence segment C) corresponds to positions 180 to 190 according to SEQ ID NO:1; and/or
sequence segment D) corresponds to positions 290 to 300 according to SEQ ID NO:1; and/or
sequence segment E) corresponds to positions 394 to 401 according to SEQ ID NO:1; and/or
sequence segment F) corresponds to positions 447 to 456 according to SEQ ID NO:1; and/or
sequence segment G) corresponds to positions 469 to 481 according to SEQ ID NO:1.

### Embodiment 4

The sucrose phosphorylase according to any of the preceding embodiments, wherein
sequence segment A) corresponds to positions 20 to 22 according to SEQ ID NO:1; and/or
sequence segment B) corresponds to positions 141 to 152 according to SEQ ID NO:1; and/or
sequence segment C) corresponds to positions 184 to 186 according to SEQ ID NO:1; and/or
sequence segment D) corresponds to positions 294 to 296 according to SEQ ID NO:1; and/or
sequence segment E) corresponds to positions 395 to 397 according to SEQ ID NO:1; and/or
sequence segment F) corresponds to positions 450 to 452 according to SEQ ID NO:1; and/or
sequence segment G) corresponds to positions 473 to 477 according to SEQ ID NO:1.

### Embodiment 5

The sucrose phosphorylase according to any of the preceding embodiments, wherein the at least one amino acid mutation is selected from the group consisting of
S21;
H142, L151;
H185;
I295;
N396;
S451;
D474, and T476.

### Embodiment 6

The sucrose phosphorylase according to any of the preceding embodiments, which comprises at least two, three or four amino acid mutations which, independently of one another in each case, are defined as in claim 1 or 2.

### Embodiment 7

The sucrose phosphorylase according to any of the preceding embodiments, which does not comprise at least one amino acid mutation selected from the group consisting of Q331E, R393N, D445P, D446G, D446T, Q460E and E485H, preferably does not comprise all these amino acid mutations.

### Embodiment 8

The sucrose phosphorylase according to any of the preceding embodiments, wherein the at least one amino acid mutation is selected from the group consisting of
S21G;
H142A, L151Y;
H185G;
I295V;
N396S;
S451T;
D474E, and T476A.

### Embodiment 9

The sucrose phosphorylase according to any of the preceding embodiments, which catalyzes the conversion of sucrose and phosphate to glucose 1-phosphate and fructose.

### Embodiment 10

The sucrose phosphorylase according to any of the preceding embodiments, which, in comparison with the sucrose phosphorylase according to SEQ ID NO:1, has

(i) an increased activity with respect to the conversion of sucrose and phosphate to glucose 1-phosphate and fructose in the presence of equimolar amounts of glucose 1-phosphate and fructose; and/or
(ii) a lower inhibition by fructose with respect to the conversion of sucrose and phosphate to glucose 1-phosphate and fructose in the presence of equimolar amounts of glucose 1-phosphate and fructose; and/or
(iii) a higher temperature stability after incubation at 58° C. for 15 min.

### Embodiment 11

The sucrose phosphorylase according to any of the preceding embodiments, which has an identity in relation to the amino acid sequence according to SEQ ID NO:4 of at least 90%.

### Embodiment 12

The sucrose phosphorylase according to embodiment 11, wherein the identity in relation to the amino acid sequence according to SEQ ID NO:4 is at least 95%.

### Embodiment 13

Use of the sucrose phosphorylase according to any of embodiments 1 to 12 for the enzymatically catalyzed conversion of sucrose and phosphate to glucose 1-phosphate and fructose.

### Embodiment 14

A method for producing glucose 1-phosphate and fructose, comprising the conversion of sucrose and phosphate under enzymatic catalysis by the sucrose phosphorylase according to any of embodiments 1 to 12.

### Embodiment 15

A method for producing cellobiose and fructose from sucrose and glucose, comprising the steps of

(a) synthesis of glucose 1-phosphate and fructose by conversion of sucrose and phosphate under enzymatic catalysis by the sucrose phosphorylase according to any of embodiments 1 to 12;
(b) synthesis of cellobiose and phosphate by reaction of the glucose 1-phosphate with glucose under enzymatic catalysis by a cellobiose phosphorylase.

Furthermore, further preferred embodiments are:

### Embodiment 16

Sucrose phosphorylase comprising an amino acid sequence which has an identity in relation to the amino acid sequence according to SEQ ID NO:1 of at least 80% and which, in comparison with SEQ ID NO:1, comprises at least two amino acid mutations
in a sequence segment B) corresponding to positions 132 to 161 according to SEQ ID NO:1; and/or
in a sequence segment E) corresponding to positions 394 to 406 according to SEQ ID NO:1; and/or
in a sequence segment G) corresponding to positions 464 to 485 according to SEQ ID NO:1.

### Embodiment 17

The sucrose phosphorylase according to embodiment 16, wherein
sequence segment B) corresponds to positions 137 to 156 according to SEQ ID NO:1; and/or
sequence segment E) corresponds to positions 394 to 401 according to SEQ ID NO:1; and/or
sequence segment G) corresponds to positions 469 to 481 according to SEQ ID NO:1.

### Embodiment 18

The sucrose phosphorylase according to embodiment 17, wherein
sequence segment B) corresponds to positions 141 to 152 according to SEQ ID NO:1; and/or
sequence segment E) corresponds to positions 395 to 397 according to SEQ ID NO:1; and/or
sequence segment G) corresponds to positions 473 to 477 according to SEQ ID NO:1.

### Embodiment 19

The sucrose phosphorylase according to embodiment 18, wherein the amino acid mutation is
H142 or L151, and/or
N396, and/or
D474 or T476.

### Embodiment 20

The sucrose phosphorylase according to embodiment 19, wherein the amino acid mutation is
H142, and/or
N396, and/or
D474.

### Embodiment 21

The sucrose phosphorylase according to embodiment 19, wherein the amino acid mutation is
L151, and/or
N396, and/or
D474.

### Embodiment 22

The sucrose phosphorylase according to any of embodiments 19 to 21, wherein the amino acid mutations are
H142A or L151Y, and/or
N396S, and/or
D474E or T476A.

The following examples illustrate the invention, but are not to be interpreted as restrictive:

### Example 1

### Engineering of the Wild-type Sucrose Phosphorylase from Bifidobacterium Adolescentis

For the engineering of the wild type of the sucrose phosphorylase from Bifidobacterium adolescentis, the following engineering goals were defined:
reduction in fructose inhibition
increase in activity in general
reduction in the Km value with regard to phosphate
reduction in G1P inhibition
maintenance of temperature stability

For the engineering, what was chosen was a semirational, iterative approach. In the first round of engineering, there was first of all, after analysis of all available data, the identification of potentially interesting positions and possible substitutions and the creation thereof as individual mutants. These were then screened in a multiparameter screening according to the desired engineering goals. Positive mutations from said first round were then combined in the following and the resultant variants were characterized.

All assays were carried out at 30° C. and pH 6.3. Commercial G1P (Sigma-Aldrich, article No. G7000) was exclusively used as G1P. The wild-type enzyme (with new codon optimization) was always co-assayed twice on each plate as comparative enzyme. All data were normalized to a comparative enzyme which was co-assayed on the same plate, making it possible to compensate for fluctuations in expression or measurements between different plates.

Overview of multiparameter screening:

| Engineering goal | Test conditions | Detection |
|---|---|---|
| Increase in | 750 mM sucrose + | ΔG1P |
| phosphorolysis | 750 mM Pi | |
| activity | | |
| Decrease in fructose | 750 mM sucrose + | ΔG1P |
| inhibition | 750 mM Pi + | |
| | 250 mM fructose | |
| Decrease in the | 750 mM sucrose + | ΔG1P |
| Pi Km value | 20 mM Pi | |
| Maintenance of | Pretreatment for | ΔG1P |
| temperature | 15 min/65° C.; | |
| stability | reaction with | |
| | 750 mM sucrose + | |
| | 750 mM Pi | |

By means of MDM analysis, 169 positions and substitutions were selected for a first round of engineering. This corresponds to about 33% of all positions, but less than 2% of all possible individual mutants. Said individual mutants were created using the AGM method (automated generation of mutants). The sequence of the wild-type gene (with codon optimization) was used as template. The variants created were expressed and were screened according to the different engineering goals. The results were normalized to the wild-type co-assayed in duplicate on each plate. It was possible to identify improved variants for all the engineering goals.

In a second round of engineering, the 7 best mutations from the first round were combined with one another (128 variants). The complex bank was created using the mutagenesis method according to WO2009/146892. 376 clones of the complex bank were picked, expressed, and screened with regard to the engineering goals (2.9-fold oversampling, screening coverage 95%). It was possible to identify improved variants for all the engineering goals.

In a third round of engineering, the hits from the second round were additionally combined with further primary hits from the 1st round (105 variants). Said variants were created, expressed and screened using the AGM method (automated generation of mutants).

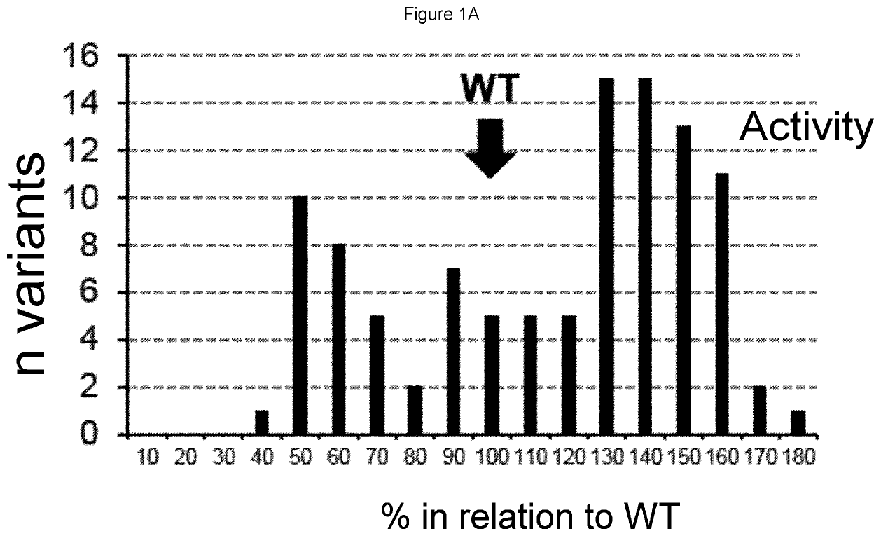

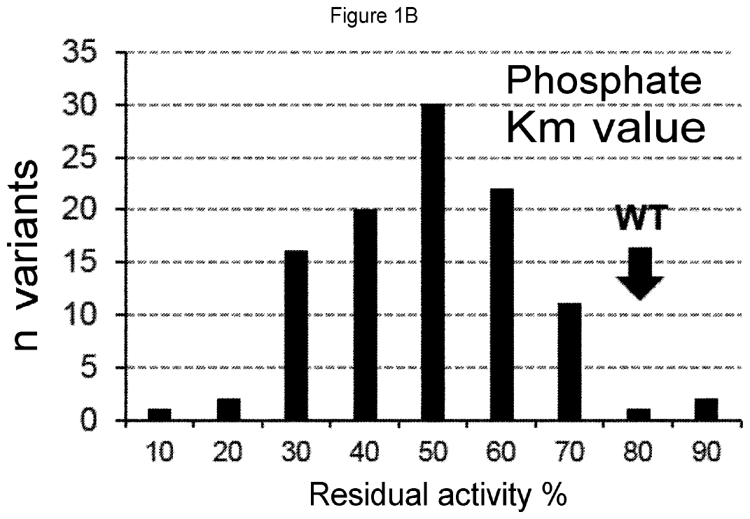

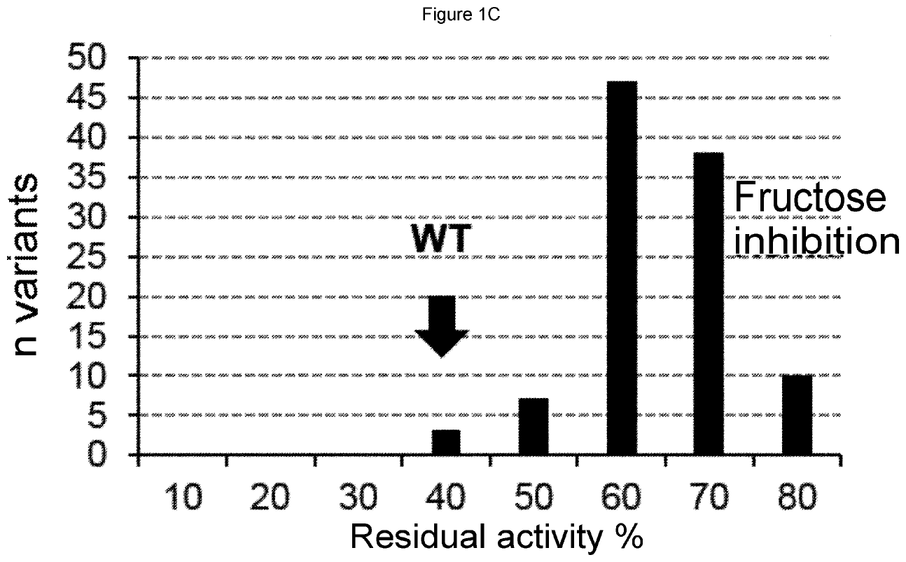

The results of the engineering with respect to phosphorolysis activity, fructose inhibition and also Km value with regard to phosphate (Pi) are depicted in comparison with the wild type (WT) in FIGS. 1A to 1C.

### Example 2

### Characterization of the 7 Best Variants

The 7 best variants from the second and third round of engineering were selected for a detailed, final characterization (SEQ ID NO:2 to 8):

| SEQ ID | | | A | B | | C | D | E | F | G | |
|---|---|---|---|---|---|---|---|---|---|---|---|
| NO. | Reference | | S21 | H142 | L151 | H185 | 1295 | N396 | S451 | D474 | T476 |
| 1 | WT | pSE014 | | | | | | | | | |
| 2 | P1-C7 | pPB115 | | A | | | | S | | E | |
| 3 | P1-C10 | pPB116 | | | Y | | | S | | E | |
| 4 | P2-A3 | pPB117 | G | | | G | V | | | E | |
| 5 | P2-B7 | pPB118 | | | | G | V | | T | E | |
| 6 | P2-B9 | pPB119 | | | | G | V | | | | A |
| 7 | PB10- | pPB111 | | | | | | S | | E | |
| | 24-1 | | | | | | | | | | |
| 8 | PB10- | pPB113 | | | | G | V | | | E | |
| | 24-3 | | | | | | | | | | |

a) Activity yield and soluble expression

The seven variants selected and the wild-type enzyme (WT) were expressed in two independent expression cultures in a shake flask, they were disrupted, and the activity yield per mL of expression culture was determined:

| SEQ ID NO: | Plasmid ID | PU/mL culture | ±s | Factor WT |
|---|---|---|---|---|
| 1 (=WT) | pSE014 | 35.4 | ±1.1 | 1.00 |
| 2 | pPB115 | 35.6 | ±2.2 | 1.00 |
| 3 | pPB116 | 46.1 | ±1.7 | 1.30 |
| 4 | pPB117 | 47.1 | ±3.0 | 1.33 |
| 5 | pPB118 | 34.9 | ±0.3 | 0.99 |
| 6 | pPB119 | 40.7 | ±1.9 | 1.15 |
| 7 | pPB111 | 46.3 | ±2.4 | 1.31 |
| 8 | pPB113 | 41.2 | ±2.5 | 1.16 |

For the assessment of soluble expression, the soluble fraction was normalized. No significant differences in the level of expression were observed between the different variants.

b) Melting curves

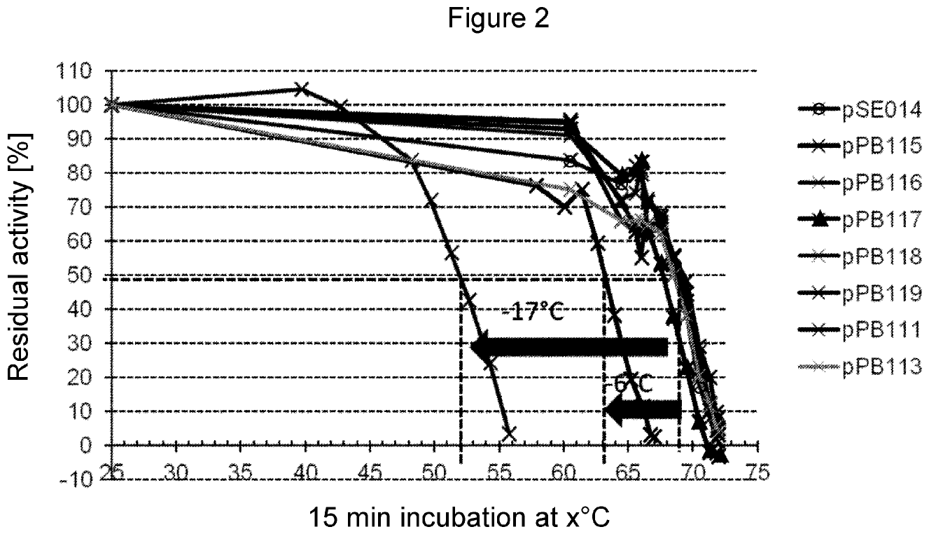

One of the engineering goals was to maintain the high temperature stability of the wild-type sucrose phosphorylase. To test the variants, the enzymes were incubated in a PCR cycler at temperatures between 40 and 72° C. for 15 min and the residual activity of the enzymes was subsequently determined. Five of the variants investigated exhibited the same temperature stability as the wild-type enzyme. The results are depicted in FIG. 2.

c) Fructose inhibition, G1P inhibition and phosphate Km value

For the assessment of possible changes in the variants with regard to fructose or G1P inhibition, the changes in the initial activities in the presence of rising fructose or G1P concentrations were measured in each case for the enzyme variants and for the wild-type enzyme. As substrate, 200 mM sucrose and phosphate were added in each case.

It became apparent that all the variants investigated had a distinctly lower inhibition in the presence of 250-750 mM fructose. Even though it was not possible to completely eliminate the fructose inhibition, the degree of improvement is notable. By contrast, in the case of the G1P inhibition, the improvements observed were not so distinct. However, the G1P inhibition of the wild type is also not so highly pronounced, like the fructose inhibition. In addition, the G1P inhibition takes effect only at G1P concentrationen above 100 mM.

Analogous to the experiments relating to enzyme inhibition, the initial activity in the presence of 10-250 mM phosphate was determined for all variants and the wild-type enzyme in order to capture changes in the Km value with regard to phosphate. None of the variants exhibited significant differences in the initial activity in comparison with the wild type at different phosphate concentrations. No variant exhibited a deterioration compared to the wild type.

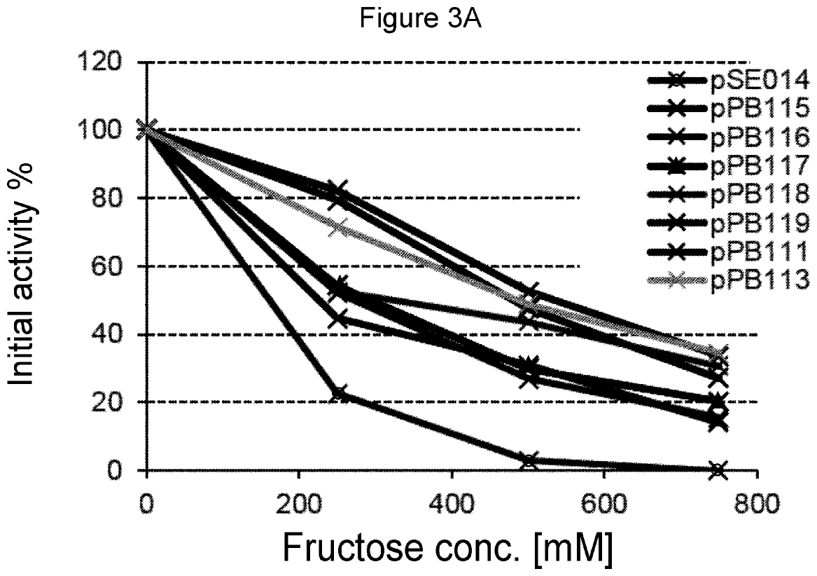

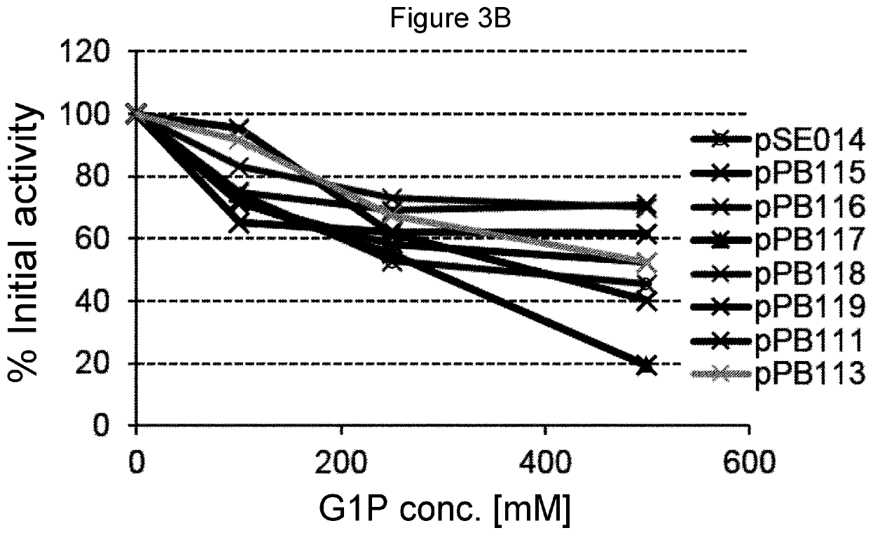

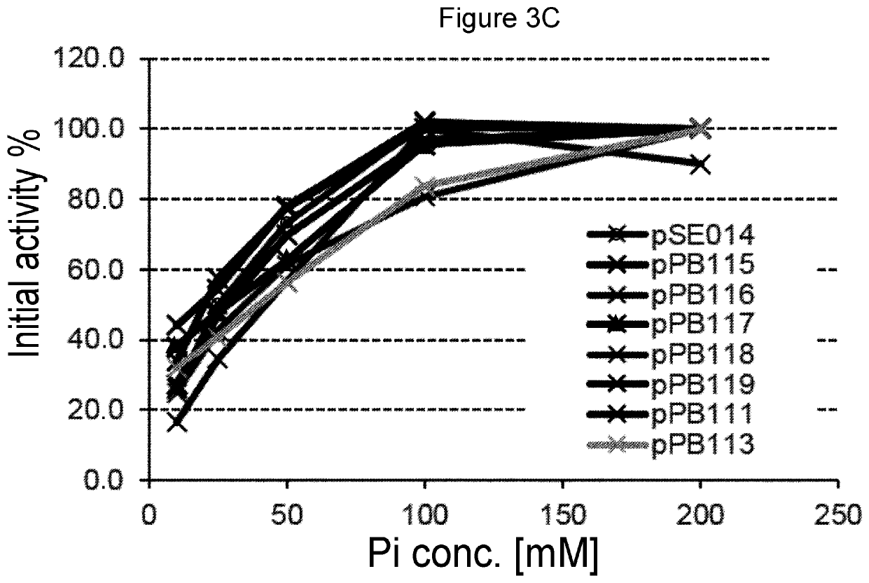

The results with respect to fructose, phosphate and glucose 1-phosphate inhibition are depicted in FIGS. 3A to 3C.

Four selected variants were expressed in a shaking flask, harvested, resuspended in 50 mM Na MES buffer, pH 6.3, and disrupted using ultrasound. Cell fragments and insoluble constituents were removed by centrifugation. The supernatant was sterile-filtered and admixed 50/50 (v/v) with sterile glycerol. The phosphorolysis activities of the formulated enzymes were determined:

| | | | Phosphorolysis |
|---|---|---|---|
| SEQ ID NO: | Reference | Volume [mL] | activity [PU/mL] |
| 8 | pPB113 | 37.5 | 408.5 |
| 4 | pPB117 | 45 | 460.2 |
| 5 | pPB118 | 47 | 384.3 |
| 6 | pPB119 | 43.5 | 444.1 |

1. A sucrose phosphorylase comprising an amino acid sequence which has an identity in relation to the amino acid sequence according to SEQ ID NO:1 of at least 80% and which, in comparison with SEQ ID NO:1, comprises an amino acid mutation in a sequence segment C) corresponding to positions 175 to 195 according to SEQ ID NO:1; and comprises an amino acid mutation in a sequence segment D) corresponding to positions 285 to 305 according to SEQ ID NO:1.

2. The sucrose phosphorylase as claimed in claim 1, wherein sequence segment C) corresponds to positions 180 to 190 according to SEQ ID NO:1; and sequence segment D) corresponds to positions 290 to 300 according to SEQ ID NO:1.

3. The sucrose phosphorylase as claimed in claim 2, wherein sequence segment C) corresponds to positions 184 to 186 according to SEQ ID NO:1; and sequence segment D) corresponds to positions 294 to 296 according to SEQ ID NO:1.

4. The sucrose phosphorylase as claimed in claim 3, wherein the amino acid mutations are H185 and position I295 or H185G and I295V.

5. (canceled)

6. The sucrose phosphorylase as claimed in any of claim 1, which, in addition to the amino acid mutations in the sequence segments C) and D), comprises at least one amino acid mutation in a sequence segment G) corresponding to positions: (i) 464 to 484 according to SEQ ID NO:1; (ii) corresponding to positions 469 to 481 according to SEQ ID NO:1; or (iii) corresponding to positions 473 to 477 according to SEQ ID NO:1.

7-8. (canceled)

9. The sucrose phosphorylase as claimed in claim 6, wherein the amino acid mutation is D474 or T476, or D474E or T476A.

10. (canceled)

11. The sucrose phosphorylase as claimed in claim 1, which, in addition to the amino acid mutations in the sequence segments C) and D) and optionally G), comprises an amino acid mutation in a sequence segment A) corresponding to positions: (i) 11 to 31 according to SEQ ID NO:1; (ii) 16 to 26 according to SEQ ID NO:1; or (iii) 20 to 22 according to SEQ ID NO:1.

12-13. (canceled)

14. The sucrose phosphorylase as claimed in claim 11, wherein the amino acid mutation is S21 or S21G.

15. (canceled)

16. The sucrose phosphorylase as claimed in claim 1, which, in addition to the amino acid mutations in the sequence segments C) and D) and optionally G), comprises an amino acid mutation in a sequence segment F) corresponding to positions: (i) 447 to 459 according to SEQ ID NO:1; or (ii) 447 to 456 according to SEQ ID NO:1.

17-18. (canceled)

19. The sucrose phosphorylase as claimed in claim 16, wherein the amino acid mutation is S451, or S451T.

20. (canceled)

21. The sucrose phosphorylase as claimed in claim 1, which does not comprise at least one amino acid mutation selected from the group consisting of Q331E, R393N, D445P, D446G, D446T, Q460E and E485H, preferably does not comprise all these amino acid mutations.

22. The sucrose phosphorylase as claimed in claim 1, which catalyzes the conversion of sucrose and phosphate to glucose 1-phosphate and fructose.

23. The sucrose phosphorylase as claimed in claim 1, which, in comparison with the sucrose phosphorylase according to SEQ ID NO:1, has
(i) an increased activity with respect to the conversion of sucrose and phosphate to glucose 1-phosphate and fructose in the presence of equimolar amounts of glucose 1-phosphate and fructose; and/or
(ii) a lower inhibition by fructose with respect to the conversion of sucrose and phosphate to glucose 1-phosphate and fructose in the presence of equimolar amounts of glucose 1-phosphate and fructose; and/or
(iii) a higher temperature stability after incubation at 58° C. for 15 min.

24. The sucrose phosphorylase as claimed in claim 1, which has an identity in relation to the amino acid sequence according to SEQ ID NO:4 of at least 90%, or at least 95%.

25. (canceled)

26. The sucrose phosphorylase as claimed in claim 1, which has an identity in relation to the amino acid sequence according to SEQ ID NO:5 of at least 90%, or at least 95%.

27. (canceled)

28. The sucrose phosphorylase as claimed in claim 1, which has an identity in relation to the amino acid sequence according to SEQ ID NO:6 of at least 90%, or at least 95%.

29. (canceled)

30. The sucrose phosphorylase as claimed in claim 1, which has an identity in relation to the amino acid sequence according to SEQ ID NO:8 of at least 90%, or at least 95%.

31-32. (canceled)

33. A method for producing glucose 1-phosphate and fructose, comprising the conversion of sucrose and phosphate under enzymatic catalysis by the sucrose phosphorylase as claimed in claim 1.

34. A method for producing cellobiose and fructose from sucrose and glucose, comprising the steps of
(a) synthesis of glucose 1-phosphate and fructose by conversion of sucrose and phosphate under enzymatic catalysis by the sucrose phosphorylase as claimed in any of claim 1;
(b) synthesis of cellobiose and phosphate by reaction of the glucose 1-phosphate with glucose under enzymatic catalysis by a cellobiose phosphorylase.
